(19) United States
(12) Patent Application Publication
(10) Pub. No.: US 2024/0336546 A1
(43) Pub. Date: Oct. 10, 2024

(54) METHOD FOR PRODUCING ALCOHOL

(71) Applicant: Resonac Corporation, Tokyo (JP)

(72) Inventors: Masafumi KOYANO, Oita-shi (JP); Gen INOUE, Oita-shi (JP); Toshihiro KIMURA, Oita-shi (JP)

(73) Assignee: Resonac Corporation, Tokyo (JP)

(21) Appl. No.: 18/571,875

(22) PCT Filed: Apr. 8, 2022
(86) PCT No.: PCT/JP2022/017416

§ 371 Date: Dec. 19, 2023

(30) Foreign Application Priority Data
Jun. 30, 2021 (JP) 2021-109181

(51) Int. Cl. C07C 29/04; B01J 8/06; B01J 21/08; B01J 23/30; B01J 37/02; C07B 41/02

(52) U.S. Cl. CPC C07C 29/04; B01J 8/065; B01J 21/08; B01J 23/30; B01J 37/0215; B01J 37/0236; C07B 41/02

(57) ABSTRACT

Provided is a method for producing an alcohol through an olefin hydration reaction using a heteropolyacid catalyst, the method enabling the catalyst to be stably used over a long period. In the olefin hydration reaction, in which the heteropolyacid catalyst is used, the pressure loss during the period of passing a feed-material gas through a catalyst layer is regulated to a certain value or less. Specifically, the method for producing an alcohol comprises using a solid-acid catalyst including a supported heteropolyacid or salt thereof, supplying water and a 2C-5C olefin to a reactor, and hydrating the olefin in a gas phase, wherein the pressure loss during the period of passing the feed-material gas through a layer of the catalyst is regulated to 350 kPa or less.

### TECHNICAL FIELD

The present invention relates to a method for producing an alcohol by a hydration reaction of an olefin using a heteropolyacid catalyst. The present invention is particularly suitable for the production of ethanol from ethylene.

### BACKGROUND

Industrial ethanol is an important industrial chemical product widely used as an organic solvent, an organic synthetic raw material, a disinfectant and an intermediate of chemicals. It is known that industrial ethanol can be obtained by a hydration reaction of ethylene in the presence of a liquid acid, such as sulfuric acid, and sulfonic acid, a zeolite catalyst, a metal oxide catalyst, such as tungsten, niobium, and tantalum, or a solid catalyst in which a heteropolyacid, such as phosphotungstic acid, and silicotungstic acid or phosphoric acid is supported on a silica carrier or a diatomite carrier, etc.

A hydration reaction of ethylene in a liquid phase using a liquid acid, such as sulfuric acid, and sulfonic acid, as a catalyst requires a post-treatment of the acid used in the reaction, and in addition, is low in activity, so that industrial utilization thereof has been limited. On the other hand, a hydration reaction of ethylene using a solid catalyst of a carrier supported type can be carried out as a gas phase reaction, and there is an advantage that the separation of a reaction product and the catalyst is easy, and the reaction can be carried out under a high temperature condition, which is advantageous in terms of reaction kinetics or a high pressure condition, which is advantageous in terms of the theory of equilibrium.

Regarding solid acid catalysts, many proposals have been made so far, and in particular, a gas phase reaction process using a solid acid catalyst in which phosphoric acid is supported on a carrier has already been industrially carried out. However, in this industrial process using a catalyst in which phosphoric acid is supported on a carrier, an efflux of phosphoric acid, which is an active component, continuously occurs, and as a result, the activity and selectivity decrease. Thus, continuous supply of phosphoric acid is required. Further, periodic maintenance of a reactor and other equipment is necessary, and thus this costs a lot to maintain the reactor and other equipment, since the effused phosphoric acid corrodes the equipment. In addition, a phosphoric acid supported catalyst is physically and chemically deteriorated by contacting with water vapor. When the phosphoric acid supported catalyst is used for a long period of time, the activity thereof decreases, and in some cases, carrier particles aggregate with each other to form a block, so that it may be extremely difficult to replace or extract the catalyst. Therefore, in a hydration reaction of ethylene, a novel carrier and a supported catalyst have been developed to solve these problems.

As a catalyst for a hydration reaction of ethylene without a risk of an efflux of a phosphoric acid, metal oxide catalysts are known, and a zeolite catalyst (Patent Literature 1), a metal oxide catalyst containing titanium oxide and tungsten oxide as essential components (Patent Literature 2), and a metal oxide catalyst containing tungsten and niobium as essential components (Patent Literature 3) are known. However, hydration reactions of ethylene using these metal oxide catalysts are less active than the case where a phosphoric acid catalyst is used, and the selectivity of the reaction is also low.

As another catalyst capable of avoiding an efflux of a phosphoric acid, a solid acid catalyst in which a heteropolyacid is supported on a carrier is known. For example, a catalyst in which a heteropolyacid is supported on fumed silica obtained by a combustion method is disclosed as a supported catalyst for the production of ethanol by a hydration reaction of ethylene having improved performance (Patent Literature 4). As a method for improving the performance of a heteropolyacid supported catalyst, the use of a catalyst in which a heteropolyacid is supported on a clay carrier treated with a thermal acid has been proposed (Patent Literature 5).

As a carrier of a supported catalyst suitable for a hydration reaction of an olefin, a silica carrier in which a pore volume, a specific surface area, and a pore diameter are specified is disclosed, and a catalyst for producing ethanol by a hydration reaction of ethylene using the silica carrier is also exemplified (Patent Literature 6).

### CITATION LIST

### Patent Literature

[PTL 1] JP H03-80136 B
[PTL 2] JP 3041414 B
[PTL 3] JP 2001-79395 A
[PTL 4] JP 3901233 B
[PTL 5] JP H08-225473 A
[PTL 6] JP 2003-190786 A

### SUMMARY OF INVENTION

### Technical Problem

Although an attempt has been made to improve the performance of a heteropolyacid supported catalyst in this way, it is desirable that a heteropolyacid supported catalyst can be stably used for a long period of time from an economic viewpoint, since the price of a heteropolyacid is expensive as compared with phosphoric acid. When a raw material gas passes through a catalyst layer, a pressure loss occurs. When the pressure loss is large, the amount of impurities, which adversely affect the productivity of the catalyst and the catalyst, increase, and therefore, it is considered that the stable use of the catalyst is adversely affected. On the other hand, until now, it has not been clarified how much a pressure loss on a catalyst layer affects long-term use of a catalyst in a hydration reaction of an olefin using a heteropolyacid catalyst.

It is an object of the present invention to provide a method which enables the stable use of a catalyst for a long period of time in the production of an alcohol by a hydration reaction of an olefin using a heteropolyacid catalyst.

### Solution to Problem

As a result of intensive studies, the present inventors have found that the pressure loss when a raw material gas passes through a catalyst layer has a large effect on deterioration of a catalyst, particularly coking, in the production of an alcohol by a hydration reaction of an olefin using a heteropolyacid catalyst. Accordingly, it has been confirmed that a catalyst can be stably used for a long period of time by keeping the pressure loss when a raw material gas passes through a catalyst layer to a specific value or less in a hydration reaction of an olefin using a heteropolyacid catalyst, and thus the present invention has been completed.

That is, the present invention relates to the following [1] to [10].

[1]

A method for producing an alcohol comprising continuously supplying a raw material gas containing water and an olefin having 2 to 5 carbon atoms to a reactor having a catalyst layer filled with a solid acid catalyst in which a heteropolyacid or a salt thereof is supported on a carrier, and subjecting them to a hydration reaction in a gas phase using the solid acid catalyst to obtain an alcohol,
wherein the pressure loss when the raw material gas passes through the catalyst layer is 350 kPa or less.
   [2]

The method for producing an alcohol according to [1], wherein the reactor is a multi-tubular reactor.

[3]

The method for producing an alcohol according to [2], wherein the solid acid catalyst is filled in tubes of the multi-tubular reactor.

[4]

The method for producing an alcohol according to [3], wherein the tubes of the multi-tubular reactor have an inner diameter of 40 mm or less.

[5]

The method for producing an alcohol according to any one of [1] to [4], wherein the superficial linear velocity of the raw material gas in the reactor is 0.1 to 1.0 m/s, and the gas space velocity of the raw material gas in the reactor is 500 to 15,000/hr.

[6]

The method for producing an alcohol according to any one of [1] to [5], wherein the conversion rate of the olefin having 2 to 5 carbon atoms is 2 to 6%.

[7]

The method for producing an alcohol according to any one of [1] to [6], wherein the partial pressure of water in the raw material gas is 0.4 to 0.6 MPa.

[8]

The method for producing an alcohol according to any one of [1] to [7], wherein the carrier is silica.

[9]

The method for producing an alcohol according to any one of [1] to [8], wherein the heteropolyacid is at least one selected from the group consisting of silicotungstic acid, phosphotungstic acid, phosphomolybdic acid, silicomolybdic acid, silicovanadotungstic acid, phosphovanadotungstic acid, and phosphovanadomolybdic acid.

The method for producing an alcohol according to any one of [1] to [9], wherein the olefin having 2 to 5 carbon atoms is ethylene and the alcohol is ethanol.

### Advantageous Effects of Invention

According to the present invention, in the production of an alcohol by a hydration reaction of an olefin using a heteropolyacid catalyst, coking of the heteropolyacid catalyst is suppressed, and the catalyst can be stably used over a long period of time.

### BRIEF DESCRIPTION OF DRAWINGS

FIG. 1A graph showing the prediction of the change with time of the by-produced acetaldehyde selectivity in the examples and comparative examples.

### DESCRIPTION OF EMBODIMENTS

Hereinafter, preferred embodiments of the present invention will be described, but the present invention is not limited to these embodiments only, and various applications can be made within the spirit and practice of the present invention.

### Solid Acid Catalyst

A solid acid catalyst of one embodiment is one in which a heteropolyacid or a salt thereof (together referred to as a “heteropolyacid catalyst”) is supported on a carrier, and is a catalyst containing a heteropolyacid or a salt thereof as a major active component of the catalyst.

### Heteropolyacid or Salt Thereof

A heteropolyacid is an acid composed of a central element and a peripheral element to which oxygen is bonded. The central element is usually silicon or phosphorus, but can be selected from any one selected from a wide variety of elements of Groups 1 to 17 of the Periodic Table of the Elements.

Examples of the central element constituting the heteropolyacid include a cupric ion; divalent ions of beryllium, zinc, cobalt and nickel; trivalent ions of boron, aluminum, gallium, iron, cerium, arsenic, antimony, phosphorus, bismuth, chromium and rhodium; tetravalent ions of silicon, germanium, tin, titanium, zirconium, vanadium, sulfur, tellurium, manganese, nickel, platinum, thorium, hafnium, cerium and other tetravalent rare earth ions; pentavalent ions of phosphorus, arsenic, vanadium, and antimony; a hexavalent ion of tellurium; and a heptavalent ion of iodine, but are not limited thereto.

Specific examples of the peripheral element include tungsten, molybdenum, vanadium, niobium, and tantalum, but are not limited thereto.

Such heteropolyacids are known as “polyoxoanions”, “polyoxometalates” or “metal oxide clusters”. The structures of some of the well-known anions are named after the researchers in this field, and for example, the Keggin structure, the Wells-Dawson structure and the Anderson-Evans-Perloff structure are known. For details, the description in “Chemistry of Polyacids” (edited by the Chemical Society of Japan, Quarterly Chemical Review No. 20, 1993) can be referred to. A heteropolyacid usually has a high molecular weight, e.g., a molecular weight in the range of 700 to 8,500, and includes not only a monomer thereof but also a dimeric complex thereof.

The salt of the heteropolyacid is not particularly limited as long as it is a metal salt or an onium salt in which some or all of the hydrogen atoms of the aforementioned heteropolyacid are substituted. Examples of the salt include metal salts of lithium, sodium, potassium, cesium, magnesium, barium, copper, gold and gallium, and onium salts, such as ammonium salts, but are not limited thereto.

A heteropolyacid has relatively high solubility in water or other polar solvents, such as oxygenated solvents, particularly when the heteropolyacid is in the form of a free acid or some types of salts. The solubility of the heteropolyacid can be controlled by selecting an appropriate counterion.

Examples of the heteropolyacid that can be used in the catalyst include:
silicotungstic acid: H₄[SiW₁₂O₄₀]·xH₂O
phosphotungstic acid: H₃[PW₁₂O₄₀]·xH₂O
phosphomolybdic acid: H₃[PMo₁₂O₄₀]·xH₂O
silicomolybdic acid: H₄[SiMo₁₂O₄₀]·xH₂O
silicovanadotungstic acid: H₄₊ₙ[SiVₙW₁₂₋ₙO₄₀]·xH₂O
phosphovanadotungstic acid: H₃₊ₙ[PVₙW₁₂₋ₙO₄₀]·xH₂O
phosphovanadomolybdic acid: H₃₊ₙ[PVₙMo₁₂₋ₙO₄₀]·xH₂O
silicovanadomolybdic acid: H₄₊ₙ[SiVₙMo₁₂₋ₙO₄₀]·xH₂O
silicomolybdotungstic acid: H₄[SiMoₙW₁₂₋ₙO₄₀]·xH₂O
phosphomolybdotungstic acid: H₃[PMoₙW₁₂₋ₙO₄₀]·xH₂O
   wherein n is an integer of 1 to 11 and x is an integer greater than or equal to 1, but are not limited thereto.

The heteropolyacid is preferably silicotungstic acid, phosphotungstic acid, phosphomolybdic acid, silicomolybdic acid, silicovanadotungstic acid, phosphovanadotungstic acid, or phosphovanadomolybdic acid, and more preferably silicotungstic acid, phosphotungstic acid, silicovanadotungstic acid, or phosphovanadotungstic acid.

There is no particular limitation on the method for synthesizing such a heteropolyacid, and any methods may be used. For example, a heteropolyacid can be obtained by heating an acidic aqueous solution (approximately pH1 to pH2) containing a salt of molybdic acid or tungstic acid and a simple oxoacid of a heteroatom or a salt thereof. A heteropolyacid compound can be isolated, for example, by crystallization separation as a metal salt from the produced aqueous heteropolyacid solution.

Specific examples of the manufacture of the heteropolyacid are described on page 1413 of “New Experimental Chemistry 8, Synthesis of Inorganic Compound (III)” (edited by the Chemical Society of Japan, published by Maruzen Co., Ltd., August 20, 1984, third edition), but are not limited thereto. The structural confirmation of the synthesized heteropolyacid can be carried out by chemical analysis, as well as X-ray diffraction, UV, or IR measurements.

Preferred examples of the salt of the heteropolyacid include lithium salts, sodium salts, potassium salts, cesium salts, magnesium salts, barium salts, copper salts, gold salts, gallium salts, and ammonium salts of the aforementioned preferred heteropolyacids.

Specific examples of the salt of the heteropolyacid include a lithium salt of silicotungstic acid, a sodium salt of silicotungstic acid, a cesium salt of silicotungstic acid, a copper salt of silicotungstic acid, a gold salt of silicotungstic acid, a gallium salt of silicotungstic acid; a lithium salt of phosphotungstic acid, a sodium salt of phosphotungstic acid, a cesium salt of phosphotungstic acid, a copper salt of phosphotungstic acid, a gold salt of phosphotungstic acid, a gallium salt of phosphotungstic acid; a lithium salt of phosphomolybdic acid, a sodium salt of phosphomolybdic acid, a cesium salt of phosphomolybdic acid, a copper salt of phosphomolybdic acid, a gold salt of phosphomolybdic acid, a gallium salt of phosphomolybdic acid; a lithium salt of silicomolybdic acid, a sodium salt of silicomolybdic acid, a cesium salt of silicomolybdic acid, a copper salt of silicomolybdic acid, a gold salt of silicomolybdic acid, a gallium salt of silicomolybdic acid; a lithium salt of silicovanadotungstic acid, a sodium salt of silicovanadotungstic acid, a cesium salt of silicovanadotungstic acid, a copper salt of silicovanadotungstic acid, a gold salt of silicovanadotungstic acid, a gallium salt of silicovanadotungstic acid; a lithium salt of phosphovanadotungstic acid, a sodium salt of phosphovanadotungstic acid, a cesium salt of phosphovanadotungstic acid, a copper salt of phosphovanadotungstic acid, a gold salt of phosphovanadotungstic acid, a gallium salt of phosphovanadotungstic acid; a lithium salt of phosphovanadomolybdic acid, a sodium salt of phosphovanadomolybdic acid, a cesium salt of phosphovanadomolybdic acid, a copper salt of phosphovanadomolybdic acid, a gold salt of phosphovanadomolybdic acid, a gallium salt of phosphovanadomolybdic acid; a lithium salt of silicovanadomolybdic acid, a sodium salt of silicovanadomolybdic acid, a cesium salt of silicovanadomolybdic acid, a copper salt of silicovanadomolybdic acid, a gold salt of silicovanadomolybdic acid, and a gallium salt of silicovanadomolybdic acid.

The salt of the heteropolyacid is preferably a lithium salt of silicotungstic acid, a sodium salt of silicotungstic acid, a cesium salt of silicotungstic acid, a copper salt of silicotungstic acid, a gold salt of silicotungstic acid, a gallium salt of silicotungstic acid; a lithium salt of phosphotungstic acid, a sodium salt of phosphotungstic acid, a cesium salt of phosphotungstic acid, a copper salt of phosphotungstic acid, a gold salt of phosphotungstic acid, a gallium salt of phosphotungstic acid; a lithium salt of silicovanadotungstic acid, a sodium salt of silicovanadotungstic acid, a cesium salt of silicovanadotungstic acid, a copper salt of silicovanadotungstic acid, a gold salt of silicovanadotungstic acid, a gallium salt of silicovanadotungstic acid; a lithium salt of phosphovanadotungstic acid, a sodium salt of phosphovanadotungstic acid, a cesium salt of phosphovanadotungstic acid, a copper salt of phosphovanadotungstic acid, a gold salt of phosphovanadotungstic acid, or a gallium salt of phosphovanadotungstic acid.

As the salt of the heteropolyacid, a lithium salt of silicotungstic acid, a cesium salt of silicotungstic acid, a lithium salt of phosphotungstic acid or a cesium salt of phosphotungstic acid is particularly preferable.

### Carrier

The heteropolyacid catalyst can be used as it is, but is preferably used as supported on a carrier. The carrier is preferably at least one selected from the group consisting of silica, diatomaceous earth, titania, activated carbon, alumina, and silica alumina, and more preferably silica.

The shape of the carrier is not particularly limited. Examples of the shape include a spherical shape, a cylindrical shape, a hollow cylindrical shape, a plate shape, an elliptical shape, a sheet shape, and a honeycomb shape. The shape is preferably spherical, cylindrical, hollow cylindrical, or elliptical, and more preferably spherical or cylindrical, in order to facilitate filling into a reactor and supporting of a catalytically active component.

Although the size of the carrier is not particularly limited, it is desirable that the size be determined by taking into consideration handling at the time of producing a solid acid catalyst on which a catalytically active component is supported or at the time of filling the catalyst, the differential pressure after filling it into a reactor, the reaction performance of the catalytic reaction, etc., since they are affected by the size. The size of the carrier is preferably 1 mm to 20 mm, and more preferably 2 mm to 10 mm, when used in a fixed bed system.

Although there is no limitation on the strength of the carrier, the crush strength of the carrier is preferably 5 N or more, and more preferably 10 N or more, since cracking or breakage of a solid acid catalyst causes an increase in the differential pressure of a reactor or occlusion of a pipe. In the present disclosure, the crush strength is a value obtained when a load is applied to a carrier by using a digital hardness meter KHT-40N type manufactured by FUJIWARA SCIENTIFIC CO., LTD., and the carrier is crushed.

Although there is no limitation on the specific surface area of the carrier, the specific surface area by the BET method is preferably 50 m²/g or more, and more preferably 100 m²/g or more, since the activity of the catalyst increases as the specific surface area increases.

There is no particular limitation on the method for supporting the heteropolyacid or the salt thereof on the carrier. In general, it can be carried out by making the carrier absorb a solution or suspension obtained by dissolving or suspending the heteropolyacid or the salt thereof in a solvent and evaporating the solvent.

The amount of the heteropolyacid or the salt thereof to be supported on the carrier can be adjusted, for example, by dissolving the heteropolyacid or the salt thereof in an amount of distilled water that corresponds to the amount of water absorbed by the carrier, and impregnating the carrier with the solution. In another embodiment, the amount of the heteropolyacid or the salt thereof to be supported on the carrier can also be adjusted by immersing the carrier in a solution of an excess amount of the heteropolyacid or the salt thereof with moderate movement, followed by filtration to remove an excess heteropolyacid or salt thereof.

The volume of the solution or suspension varies depending on the carrier used, the supporting method, etc. By placing a carrier in which the heteropolyacid or the salt thereof is impregnated in a heating oven for several hours to evaporate a solvent, a solid acid catalyst supported on the carrier can be obtained. The drying method is not particularly limited, and various methods, such as a stationary method, and a belt conveyor method, can be used. The amount of the heteropolyacid or the salt thereof supported on the carrier can be accurately measured by chemical analysis, such as ICP and XRF.

The amount of the heteropolyacid or the salt thereof supported on the carrier is preferably 10 to 300 parts by mass, and more preferably 20 to 200 parts by mass, in terms of the total mass of the heteropolyacid and the salt thereof with respect to 100 parts by mass of the carrier.

### Method for Producing Alcohol

Next, a method for producing an alcohol by a hydration reaction of an olefin using a heteropolyacid catalyst will be described. An alcohol can be obtained by supplying a raw material gas containing water and an olefin having 2 to 5 carbon atoms to a reactor having a catalyst layer filled with a solid acid catalyst in which a heteropolyacid or a salt thereof is supported on a carrier, and subjecting them to a hydration reaction in a gas phase using the solid acid catalyst.

A specific example of the alcohol production reaction by the hydration reaction of an olefin having 2 to 5 carbon atoms is represented by the following reaction formula (1):

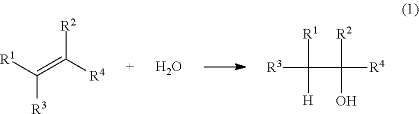

wherein R¹ to R⁴ each independently represent a hydrogen atom or an alkyl group having 1 to 3 carbon atoms, and the sum of the carbon atoms of R¹ to R⁴ is 0 to 3.

There is no particular limitation on the olefin having 2 to 5 carbon atoms which can be used. Examples of the olefin having 2 to 5 carbon atoms include ethylene, propylene, n-butene, isobutene, pentene and a mixture of two or more thereof. Among them, ethylene is more preferable.

Although there is no limitation on the use ratio of the olefin and water, the molar ratio of the olefin to water is preferably water/olefin=0.01 to 2.0, and more preferably water/olefin=0.1 to 1.0, since the concentration dependence of the olefin on the reaction rate is large and the energy cost of the alcohol production process increases when the water concentration is high.

There is no limitation on the mode of the hydration reaction of an olefin using the heteropolyacid catalyst, and any of the reaction modes can be used. The preferred mode is a fixed-bed, in terms of favorable reaction efficiency and the least energy required for separation from the catalyst. A solid acid catalyst of one embodiment is filled in a fixed-bed reactor to form a catalyst layer.

As the fixed-bed reactor, a multi-tubular reactor, which has high heat removal efficiency, is preferable. A reactor having poor heat removal efficiency makes the temperature difference in a catalyst layer larger, which is not preferable. A multi-tubular reactor comprises a plurality of tubes as a reaction tube, and the solid acid catalyst can be filled in these tubes to form a catalyst layer.

The tube of the multi-tubular reactor has an inner diameter of preferably 40 mm or less, from the viewpoint of heat removal efficiency. In addition, from the viewpoint of allowing a raw material gas to flow uniformly through each tube, the inner diameter and the length of each tube are preferably uniform. The number of the tubes depends on the size of a reactor, but can be a few to several thousands.

When a fixed-bed reactor other than a fixed-bed multi-tubular reactor is used, the temperature difference in a catalyst layer can be reduced by alternately combining small adiabatic reactors and heat exchangers in series.

As a coolant used for heat removal of a reactor, it is preferable that liquid phase water be used. When liquid phase water is used, heat of a process fluid in a reactor can be efficiently removed by vaporization heat of the coolant.

### Reaction Conditions

The superficial linear velocity of a raw material gas flowing in a reactor is preferably 0.1 to 1.0 m/s. When the superficial velocity is 0.1 m/s or more, an overall heat transfer coefficient is not excessively reduced, and the heat removal efficiency can be maintained. When the superficial velocity is 1.0 m/s or less, the pressure loss in the reactor is not excessively increased, and therefore the catalyst is less likely to be crushed or the load on a circulating gas compressor is less likely to increase. The superficial linear velocity is obtained by the following formula (1).

$$\\ \begin{matrix}( superficial linear velocity [ m / s ] ) = ( actual gas flow rate [ {m}^{3} / s ] ) / ( reactor cross ‐ sectional area [ {m}^{2} ] ) & ( 1 )\end{matrix}$$

The gas space velocity in the reactor is not particularly limited, but is preferably 500 to 15,000/hr, and more preferably 1,000 to 4,000/hr, from the viewpoint of energy and reaction efficiency. When the gas space velocity is 500/hr or more, the amount of the catalyst used can be effectively reduced, and when the gas space velocity is 15,000/hr or less, the amount of gas circulation can be reduced, so that the production of an alcohol can be more efficiently carried out within the above ranges. The gas space velocity is obtained by the following formula (2).

$$\\ \begin{matrix}( Gas space velocity [ / s ] ) = ( Gas flow rate [ {Nm}^{3} / s ] ) / ( Catalyst loading [ {m}^{3} ] ) & ( 2 )\end{matrix}$$

There is no limitation on the reaction pressure in the hydration reaction of an olefin using the heteropolyacid catalyst. Since the hydration reaction of an olefin is a reaction in which the number of molecules decreases, it is generally advantageous to proceed at high pressure. The reaction pressure is preferably 0.5 to 7.0 MPaG, and more preferably 1.5 to 4.0 MPaG. “G” means a gauge pressure. When the reaction pressure is 0.5 MPaG or more, a satisfactory reaction rate can be obtained. When the reaction pressure is 7.0 MPaG or less, it is not necessary to provide equipment as countermeasures for condensation of an olefin and in relation to evaporation of an olefin, and equipment for high pressure gas safety, and it is possible to further reduce costs regarding energy.

The partial pressure of water in a raw material gas is preferably 0.4 to 0.6 MPa.

The pressure loss when a raw material gas passes through a catalyst layer of a reactor is a differential pressure between the static pressure of the raw material gas at the inlet of the catalyst layer and the static pressure of the gas (reaction gas) after the reaction at the outlet of the catalyst layer. The pressure loss in the catalyst layer can be measured by measuring the differential pressure with a differential pressure gauge.

In one embodiment, the value of the pressure loss when a raw material gas passes through a catalytic layer in a reactor is 350 kPa or less, more preferably 200 kPa or less, further preferably 50 kPa or less. When the value of the pressure loss is 350 kPa or less, it is possible to reduce the difference in partial pressures of the respective raw material components in the vertically direction of the catalytic layer.

In the case of using a multi-tubular reactor, the pressure loss is a differential pressure between a static pressure of a chamber that distributes a raw material gas to a plurality of tubes, which are reaction tubes, and a static pressure of a chamber that collects the reaction gas from the plurality of tube outlets.

The reaction temperature of the hydration reaction of an olefin using the heteropolyacid catalyst is not particularly limited, and the reaction can be carried out at a wide range of temperatures. In view of the thermal stability of the heteropolyacid or the salt thereof and the temperature at which water, one of the raw materials, does not condense, the preferred reaction temperature is 100 to 550° C., and more preferably 150 to 350° C.

The hydration reaction of an olefin using the heteropolyacid catalyst is an equilibrium reaction, and the conversion rate of the olefin will be an equilibrium conversion rate at most. For example, the equilibrium conversion rate in the production of ethanol by the hydration of ethylene is calculated to be 7.5% at a temperature of 200° C. and a pressure of 2.0 MPaG. Therefore, in the method for producing an alcohol by the hydration of an olefin, a maximum conversion rate is determined by the equilibrium conversion rate, and as can be seen in an example of ethylene, the hydration reaction of an olefin tends to have a small equilibrium conversion rate, and thus it is strongly required in the industry to carry out the hydration reaction of an olefin with high efficiency under mild conditions.

For the above reasons, the olefin conversion rate in the olefin hydration reaction is preferably 2 to 6%. When the conversion rate is 2% or more, the circulation amount of unreacted ethylene can be reduced, which is economically advantageous. When the conversion rate is 6% or less, the difference from the equilibrium conversion rate can be made to the extent necessary for maintaining the reaction rate, and severe conditions, such as high pressure are not essential. Therefore, it is advantageous in terms of economy and equipment.

In the hydration reaction of an olefin using the heteropolyacid catalyst, loss of the olefin can be reduced by recycling any unreacted olefin into a reactor. There is no limitation on the method for recycling the unreacted olefin into the reactor, and the olefin may be isolated and recycled from a process fluid coming out of the reactor, or may be recycled together with other inert components. Typically, an industrial grade olefin often contains a very small amount of paraffin. Therefore, for example, when ethylene containing ethane is used and the unreacted ethylene is recycled to the reactor, it is desirable to purge a portion of the recovered reaction gas, i.e., ethylene gas, out of the system, in order to prevent concentration and accumulation of ethane.

In the hydration reaction of an olefin using the heteropolyacid catalyst, a dehydration reaction may occur between alcohols, which is a product, to generate an ether compound as a by-product. For example, when ethanol is obtained by the hydration of ethylene, diethyl ether is by-produced. It is considered that this diethyl ether is generated by a dehydration reaction of two molecules of ethanol, and when ethanol is produced by the hydration reaction of ethylene, the reaction yield is remarkably lowered.

However, by recycling the by-produced diethyl ether into the reactor, diethyl ether is converted into ethanol, so that ethanol can be produced from ethylene with extremely high efficiency. Although there is no particular limitation on the method for recycling the by-produced ether compound into the reactor, there are, for example, a method including isolating an ether compound from components distilled from the reactor and recycling the ether compound into the reactor, and a method including recycling the ether compound into the reactor as a gas component together with an unreacted olefin.

In the hydration reaction of an olefin using the heteropolyacid catalyst, the produced alcohol in a state of being dissolved in a large amount of water which has not been converted as a reaction raw material is sent to a separation and purification step together with other by-products. In the separation and purification step, the alcohol, water, and the other by-products are separated, and an alcohol having a purity equal to or higher than a certain level by the purification is obtained as a product.

At this time, the water simultaneously obtained may be disposed of as waste water, but from the viewpoint of impact or load on the environment, it is desirable to recycle it in the process and use it again as a raw material for the reaction. There is no limitation on the type and number of apparatuses in the separation and purification step, and a distillation apparatus or a membrane separation apparatus may be used, and different apparatuses can be used in combination if necessary.

### EXAMPLES

Although the present invention will be further described with reference to the following Examples and Comparative Examples, these examples illustrate the summary of the present invention, and the present invention is not limited to these examples.

### 1. Preparation of Silica Carrier

40 parts by mass of fumed silica: Aerosil (trademark) 300 (Nippon Aerosil Co., Ltd.), 60 parts by mass of silica gel: CARIACT G6 (Fuji Silysia Chemical Ltd.), and 40 parts by mass of colloidal silica: Snowtex O (Nissan Chemical Corporation) were kneaded by a kneader, and then water, methyl cellulose: METOLOSE (trademark) SM-4000 manufactured by Shin-Etsu Chemical Co., Ltd., as an additive, and Cerander (trademark) YB-132A manufactured by Yuken Industry Co., Ltd., as a resin-based binder were added in an appropriate amount, and the mixture was further kneaded to prepare a kneaded material.

Next, the kneaded material was put into an extruder, to which a die having a circular hole of 3 mmo at a tip thereof was attached. The kneaded material was extruded from the extruder, and the extruded intermediate material was cut into 3 mm with a cutter to obtain a cylindrical shaped body before calcination. The obtained shaped body before calcination was formed into a spherical shape by Marumerizer (trademark), and preliminary dried at 70° C. for 24 hours or more. Next, the obtained dried shaped body was subjected to a calcining treatment at 820° C. under an air atmosphere, and cooled to obtain a silica carrier.

### Water Absorption Rate Measurement of Silica Carrier

The water absorption rate of the obtained silica carrier was measured by the following methods.
(1) About 5 g of the carrier was weighed on a balance (W1 (g)), and placed in a 100 ml beaker.
(2) About 15 mL of pure water (ion-exchanged water) was added to the beaker so as to completely cover the carrier.
(3) The carrier was allowed to stand for 30 minutes.
(4) Pure water was drained by putting the carrier and pure water on a wire mesh, and the carrier was collected.
(5) Water adhering to the surface of the carrier was removed by lightly pressing with a paper towel until there is no gloss on the surface.
(6) The total mass of the carrier and pure water obtained in (5) was measured (W2 (g)).
(7) The water absorption rate of the carrier was calculated by the following formula.

$$\\ Water absorption rate ( g ‐ water / g ‐ carrier ) ( \% ) = ( W 2 - W 1 ) / W 1 \times 100$$

Therefore, the water absorption amount (g) of the carrier can be calculated by “the water absorption rate (g-water/g-carrier) (%) of the carrier×the mass of the carrier used (g)”.

### 2. Preparation of Solid Acid Catalyst in Which Heteropolyacid Is Supported on Silica Carrier

In a 100 ml beaker, 40.7 g of a commercially available Keggin silicotungstic acid 26 hydrate (H₄SiW₁₂O₄₀·26H₂O, Nippon Inorganic Chemical Industry Co., Ltd.) was weighed, a small amount of distilled water was added to solve silicotungstic acid, and then the solution was transferred to a 200 mL graduated cylinder. Then, distilled water was added so that the liquid amount of the silicotungstic acid solution in the graduated cylinder was 95% of the water absorption rate of a silica carrier to be used, and the mixture was stirred so that the entire mixture was uniform. After stirring, the aqueous solution of silicotungstic acid was transferred to a 200 mL volumetric flask, and then weighed 100 mL of the silica carrier was put into the 200 mL volumetric flask, and the contents of the volumetric flask were mixed so that the aqueous solution of silicotungstic acid contacted the entire carrier, and silicotungstic acid was supported on the silica carrier. The silica carrier on which silicotungstic acid was supported was transferred to a porcelain dish, air-dried for one hour, and then dried for 5 hours in a hot air dryer adjusted to 150° C. After drying, the carrier was transferred into a desiccator and cooled to room temperature to obtain a heteropolyacid catalyst (solid acid catalyst).

### 3. Hydration Reaction of Ethylene

A double tube reactor (SUS316, inner diameter 34 mm, length 6.7 m) of a size assuming an actual multi-tubular reactor was filled with a predetermined amount of the heteropolyacid catalyst (solid acid catalyst). Steam condensate (hot water at high pressure) was pumped to the shell side of the double tube reactor, and the catalyst layer on the tube side was adjusted to a predetermined temperature. After the reactor was pressurized to a predetermined pressure, a predetermined amount of water vaporized by an evaporator and a predetermined amount of ethylene were introduced into the reactor.

After the start of the reaction, hot water was circulated to the shell side of the double tube reactor to remove the reaction heat. A reaction gas passed through the reactor was cooled, and a condensed reaction liquid, a reaction gas passed through a scrubber after removing the condensate (reaction liquid), and washing water from the scrubber were sampled for a certain period of time, respectively. The sampled reaction liquid, reaction gas, and washing water were analyzed using a gas chromatography analyzer and a Karl Fischer analyzer by a method described later, and the reaction results were calculated.

### 4. Analysis of Reaction Gas

The sampled reaction gas was analyzed by using a gas chromatography apparatus (apparatus name: 7890, Agilent Technologies Japan, Ltd.), and a system program based on a plurality of columns and two detectors under the following conditions:

### Gas Chromatography Conditions:

Oven: kept at 40° C. for 3 minutes, then raised to 200° C. at 20° C./min
Carrier gas: helium
Split ratio: 10:1

### Columns Used: Agilent Technologies Japan, Ltd.

HP-1:2 m
GasPro: 30 m×320 μm
DB-624:60 m×320 μm×1.8 μm

### Detectors:

Front detector: FID (heater: 230° C., hydrogen flow rate 40 mL/min, air flow rate 400 L/min)
Back detector: FID (heater: 230° C., hydrogen flow rate 40 mL/min, air flow rate 400 L/min)
Aux detector: TCD (heater: 230° C., reference flow rate 45 mL/min, make-up flow rate 2 mL/min)

### 5. Analysis of Reaction Liquid and Wash Water

The sampled reaction liquid and wash water were analyzed by using a gas chromatography apparatus (apparatus name: 6850, Agilent Technologies Japan, Ltd).
Columns used: PoraBONDQ 25 m×0.53 mm ID×10 μm
Oven temperature: kept at 100° C. for 2 minutes, then raised to 240° C. at 5° C./min.
Injection temperature: 250° C.
Detector temperature: 300° C.
Detector: FID

The concentration of water in the reaction liquid was analyzed by a Karl Fischer analyzer (Mitsubishi Chemical Co., Ltd.).

### 6. Calculation of Reaction Results

Ethylene conversion rate and acetaldehyde selectivity were calculated by the following formulae.

$$\\ Ethylene conversion rate ( mol \% ) = ( \begin{matrix}Number of moles of reacted ethylene / \\ Number of moles of fed ethylene\end{matrix} ) \times 100$$

$$\\ Acetaldehyde selectivity ( mol \% ) = ( \begin{matrix}Number of moles of produced acetaldehyde / \\ Number of moles of fed ethylene\end{matrix} ) \times 100$$

### Example 1

A double tube reactor (SUS316, inner diameter 34 mm, length 6.7 m) was charged with 3.1 L of the heteropolyacid catalyst (solid acid catalyst). At this time, the height of the catalyst layer was 3.6 m. The inside of the reactor was replaced with nitrogen gas. Then, the reactor was pressurized to 2.4 MPaG. Then, the reactor was heated to 180° C., and at a stage where the temperature was stable, amounts of water vaporized by an evaporator and ethylene so that the molar ratio of water to ethylene was 0.3, diethyl ether in an amount balanced before and after the reactor (3% by volume), and nitrogen gas (13% by volume) as an inert gas were supplied to the reactor at a GHSV (gas space velocity) of 3000/hr to carry out a hydration reaction of ethylene.

The pressure of the raw material gas at the inlet of the reactor was maintained at 2.4 MPaG by the automatic control by a pressure regulating valve. After the feed of the raw material gas, the average temperature of the catalyst layer was adjusted so that the conversion rate of ethylene was 4.1% after the temperature was stable. The average temperature was 190° C. The superficial linear velocity of the raw material gas was 0.20 m/s. The partial pressure of water in the raw material gas was 0.48 MPa.

The pressure loss, which is the pressure difference between the raw material gas at the inlet of the reactor (i.e., the inlet of the catalyst layer) and the reaction gas at the outlet of the reactor (the outlet of the catalyst layer), was 27 kPa.

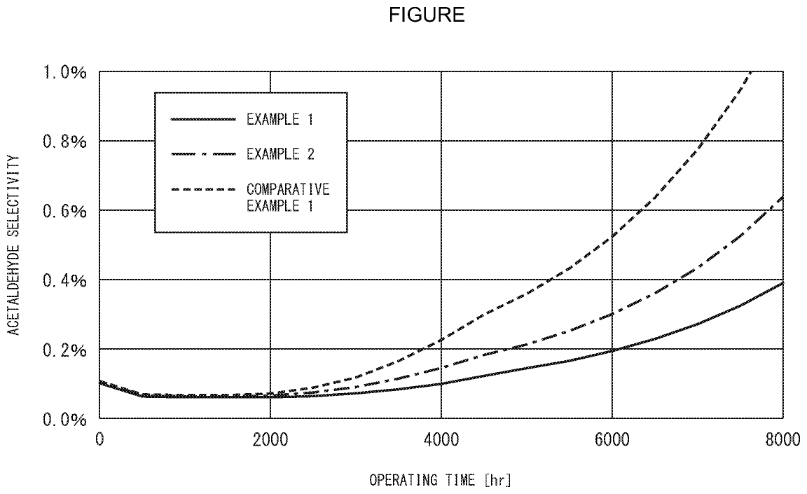

A gas passed through the reactor was cooled, and sampling of a condensed reaction liquid, a washing liquid, and a reaction gas from which the reaction liquid was removed was carried out. The obtained reaction liquid, the washing liquid, and the reaction gas were analyzed by the above-described method. Reaction results of the catalyst were calculated based on the masses, the gas flow rates, and analysis results. A continuous test for 400 hours was carried out, and by fitting the stable reaction results to the existing long-term life test results, the transition of reaction results for one year (change with time in a selectivity of acetaldehyde, which is a representative by-product) was predicted. The results are shown in Table 1 and FIG. 1.

### Example 2

The hydration reaction of ethylene was carried out in the same manner as in Example 1, except that the double tube reactor was charged with 5.7 L of the heteropolyacid catalyst (solid acid catalyst). The average temperature of the catalyst layer was adjusted so that the conversion rate of ethylene was 4.1% The average temperature was 190° C. In the same manner as in Example 1, GHSV (gas space velocity) was 3000/hr. The superficial linear velocity of the raw material gas was 0.37 m/s. The pressure loss of the raw material gas was 200 kPa. Further, in the same manner as in Example 1, the reaction results were calculated, and the transition of reaction results for one year (change with time in a selectivity of acetaldehyde, which is a representative by-product) was predicted. The results are shown in Table 1 and FIG. 1.

### Comparative Example 1

The hydration reaction of ethylene was carried out in the same manner as in Example 1, except that the double tube reactor was charged with 6.6 L of the heteropolyacid catalyst (solid acid catalyst). The pressure loss of the raw material gas was 400 kPa. The average temperature of the catalytic layer was 190° C., GHSV (gas space velocity) was 3000/hr, and the superficial linear velocity was 0.43 m/s. Further, in the same manner as in Example 1, the reaction results were calculated, and the transition of reaction results for one year (change with time in a selectivity of acetaldehyde, which is a representative by-product) was predicted. The results are shown in Table 1 and FIG. 1.

TABLE 1

| | Pressure | Ethylene | Ethanol | Acetaldehyde |
|---|---|---|---|---|
| | loss | conversion | STY | selectivity |
| | [kPa] | rate [%] | [kg/m³/h] | (stable at 400 hrs) |
| Ex. 1 | 27 | 4.1 | 153 | 0.060% |
| Ex. 2 | 200 | 4.1 | 153 | 0.063% |
| Comp. Ex. 1 | 400 | 4.1 | 153 | 0.067% |

### Reaction Results

In Comparative Example 1, the pressure loss of the raw material gas when passing through the catalyst layer is 400 kPa, and the selectivity of acetaldehyde, which is a by-product, is high. When acetaldehyde is generated, acetaldehyde polymerizes on a catalyst surface to form a coke, which reduces the reaction activity of the catalyst or deteriorates the selectivity thereof, so that the catalyst cannot be used stably for a long period of time. From the results of Examples 1 and 2, it can be seen that the acetaldehyde selectivity is reduced by reducing the pressure loss when the raw material gas passes through the catalyst layer, and these conditions are advantageous for stable use of the catalyst over a long period of time.

### Prediction of Reaction Results for One Year

For Examples 1 and 2, it can be said that a low acetaldehyde selectivity can be maintained throughout a year, and an alcohol can be stably produced over a long period of time. In Comparative Example 1, the increase rate in the acetaldehyde selectivity is large, and the acetaldehyde selectivity exceeds 1%, which is an indication that a catalyst can be used stably.

### INDUSTRIAL APPLICABILITY

The present invention is industrially useful in that, in the production of an alcohol by a hydration reaction of an olefin using a heteropolyacid catalyst, a catalyst can be stably used for a long period of time by reducing a pressure loss when a raw material gas passes through a catalyst layer, which is economically advantageous.

1. A method for producing an alcohol comprising continuously supplying a raw material gas containing water and an olefin having 2 to 5 carbon atoms to a reactor having a catalyst layer filled with a solid acid catalyst in which a heteropolyacid or a salt thereof is supported on a carrier, and subjecting them to a hydration reaction in a gas phase using the solid acid catalyst to obtain an alcohol,
wherein the pressure loss when the raw material gas passes through the catalyst layer is 350 kPa or less.

2. The method for producing an alcohol according to claim 1, wherein the reactor is a multi-tubular reactor.

3. The method for producing an alcohol according to claim 2, wherein the solid acid catalyst is filled in tubes of the multi-tubular reactor.

4. The method for producing an alcohol according to claim 3, wherein the tubes of the multi-tubular reactor have an inner diameter of 40 mm or less.

5. The method for producing an alcohol according to claim 1, wherein the superficial linear velocity of the raw material gas in the reactor is 0.1 to 1.0 m/s, and the gas space velocity of the raw material gas in the reactor is 500 to 15,000/hr.

6. The method for producing an alcohol according to claim 1, wherein the conversion rate of the olefin having 2 to 5 carbon atoms is 2 to 6%.

7. The method for producing an alcohol according to claim 1, wherein the partial pressure of water in the raw material gas is 0.4 to 0.6 MPa.

8. The method for producing an alcohol according to claim 1, wherein the carrier is silica.

9. The method for producing an alcohol according to claim 1, wherein the heteropolyacid is at least one selected from the group consisting of silicotungstic acid, phosphotungstic acid, phosphomolybdic acid, silicomolybdic acid, silicovanadotungstic acid, phosphovanadotungstic acid, and phosphovanadomolybdic acid.

10. The method for producing an alcohol according to claim 1, wherein the olefin having 2 to 5 carbon atoms is ethylene and the alcohol is ethanol.
